(19) United States
(12) Patent Application Publication
(10) Pub. No.: US 2012/0289893 A1
(43) Pub. Date: Nov. 15, 2012

(54) SUCTION CATHETER CONTROLLER AND SUCTION CATHETER ASSEMBLY UTILIZING THE SAME

(75) Inventor: CHENG-HUANG CHUNG, Taipei City (TW)

(73) Assignee: LILY MEDICAL CORPORATION, Miao-Li Hsien (TW)

(21) Appl. No.: 13/105,832

(22) Filed: May 11, 2011

(51) Int. Cl. A61M 1/00

(52) U.S. Cl. 604/30; 604/540

(57) ABSTRACT

A suction catheter assembly is disclosed, which includes a manifold member, an engaging structure, a suction catheter, and a controller. The controller includes a housing, an elastic member, a hollow valve body, an actuating piston, and a cap. The housing has an inlet portion and an outlet portion. The housing has a sealed portion formed on one end thereof. The actuating piston projects through the hollow valve body and abuts to the cap and the elastic member on opposite ends. When the hollow valve body is at closed position, fluid flow is cut off between the inlet and outlet portions. When the hollow valve body is at open position, the fluid flow is allowed. The suction catheter assembly has better air tightness.

### BACKGROUND OF THE INVENTION

1. Field of the Invention

The instant disclosure relates to a suction catheter controller and a suction catheter assembly utilizing the same; more particularly, to a suction catheter controller and a suction catheter assembly having the same having improved sealing properties.

2. Description of Related Art

In medical care, patients in critical conditions are often unable to clear their own secretions in the respiratory tract. Doctors usually use suction catheters to help removing the fluids from such patients. The purpose of suctioning is to keep the airways clear of secretions. Since the suction catheter must be inserted into the respiratory tract or the artificial airway (e.g. tracheal tube), each suctioning procedure exposes the patient to the risk of infection. Other potential complications may include irregular heart beats, slow heart rates, and heart failures. As shown in FIG. 1, a conventional respiratory suctioning device 1′ includes a vacuum generator (not shown), a controller 10′ connected to the vacuum generator, and a suction catheter 20′ connected to the controller 10′ and for inserting into a patient's respiratory tract or artificial airway. By turning on the vacuum generator, a negative pressure is created inside the controller 10′ and the suction catheter 20′. The fluids are drained accordingly by the suction catheter 20′ into the controller 10′. The controller 10′ is usually disposed at one end of the respiratory suctioning device F. The controller 10′ comprises a housing 101′, a bottom cap 102′ disposed at one end of the housing 101′, and a top cap 103′ disposed at opposite end that can be pressed. However, the bottom cap 102′ is not tightly sealed to the housing 101′. When handling the respiratory suctioning device 1′, the care giver may come in contact with the contaminated fluids and be exposed to cross-contamination. The insecure sealing makes the controller 10′ a medium of potentially spreading the infections. In addition, the suction catheter 20′ is not connected to a check valve (not shown) to prevent the backflow of the fluids. Therefore, the patient may suffer discomfort and inconvenience in using the respiratory suctioning device 1′.

To address the above issues, the inventor proposes the following solutions.

### SUMMARY OF THE INVENTION

The instant disclosure provides a controller for the suction catheter and a suction catheter assembly having the same. The controller has excellent air tightness and operation characteristics, along with being easy to assemble and environmental friendly.

According to one aspect of the instant disclosure, a controller for the suction catheter comprises: a housing having an inlet portion and an outlet portion, wherein the housing has a coaxial tubular inner housing portion passingly bridging the inlet and outlet portions; an elastic member disposed within the tubular wall, wherein one end of the elastic member abuts the inner surface of the housing; a hollow valve body, wherein the hollow valve body is movably disposed within the tubular inner housing portion at closed or open position, wherein at the closed position, fluid flow is cut off between the inlet and outlet portions, wherein at the open position, flow is allowed; a actuating piston that projects through the hollow valve body, wherein one end of the actuating piston abuts to the elastic member; a cap disposed on the housing, wherein an opening is formed on the cap to engage the actuating piston.

According to another aspect of the instant disclosure, a suction catheter assembly comprises: a manifold member having a tubular arm and a sleeve part, wherein the tubular arm is piped to the sleeve, and the sleeve receives a switch valve having a thru hole formed thereon, with the switch valve being rotatably disposed in between the open and closed positions, in which the thru hole is in line with the arm when the switch valve is at the open position, while the thru hole is normal to the arm at the closed position to block the flow; an engaging structure disposed at one end of the arm, wherein the engaging structure has at least one breathing tube connector piped to an injection port; a suction catheter disposed at opposite end of the arm; and a controller disposed at one end of the suction catheter opposite of the manifold member. The controller comprises a housing, an elastic member, a actuating piston, a hollow valve body, and a cap. The housing has an inlet portion and an outlet portion, wherein the housing also has a coaxial tubular inner housing portion passingly bridging the inlet and outlet portions. The elastic member is disposed within the tubular wall, wherein one end of the elastic member abuts the inner surface of the housing. The hollow valve body has a tubular body, which is movably disposed in the tubular wall at a closed or open position. At the closed position, the hollow valve body cuts off the flow between the inlet and the outlet portions. At the open position, fluid flow is allowed. The actuating piston is projected through the hollow valve body, wherein one end of the actuating piston abuts to the elastic member. The cap is disposed on the housing, wherein an opening is formed on the cap to engage the actuating piston.

The instant disclosure has the following advantages. The controller only requires the elastic member, the actuating piston, and the hollow valve body to be assembled into the housing, followed by disposing the cap. The assembling process is easy for efficient manufacturing. Also, the controller has a sealed portion opposite of the cap, which significantly reduces the potential of exposing to infections for protecting the care giver. Furthermore, the actuating piston, the hollow valve body, and the cap work together in providing directional control over the catheter assembly. In comparing to conventional design, the actuating piston and the elastic member have a greater contact area with each other. Thus, less human effort is spent to operate the controller.

In order to further appreciate the characteristics and technical contents of the instant disclosure, references are hereunder made to the detailed descriptions and appended drawings in connection with the instant disclosure. However, the appended drawings are merely shown for exemplary purposes, rather than being used to restrict the scope of the instant disclosure.

### BRIEF DESCRIPTION OF THE DRAWINGS

FIG. 1 is a perspective view of a respiratory suctioning device of the related art.

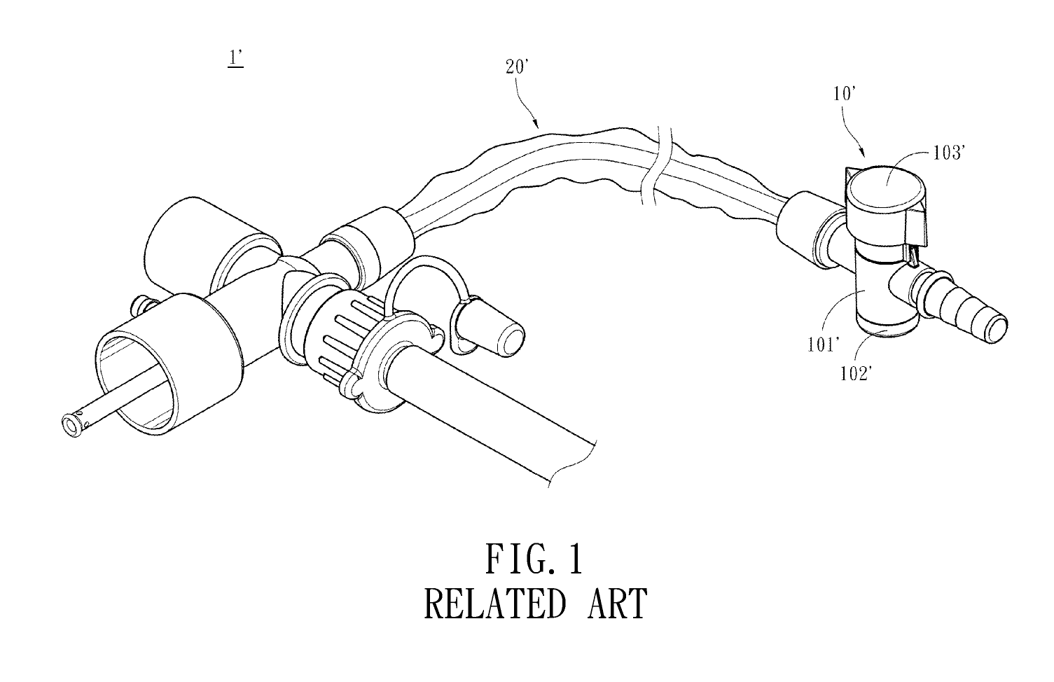

FIG. 2 is an exploded view of a controller for the suction catheter of the instant disclosure.

FIG. 3 is a sectional view of the controller showing a hollow valve body at closed position.

FIG. 4 is a sectional view of the controller showing the hollow valve body at open position.

FIG. 5 is a transparent view of the controller showing the hollow valve body at closed position.

FIG. 6 is an assembled view of the suction catheter assembly of the instant disclosure.

FIG. 7 is an exploded view for part of the catheter assembly.

### DETAILED DESCRIPTION OF PREFERRED EMBODIMENTS

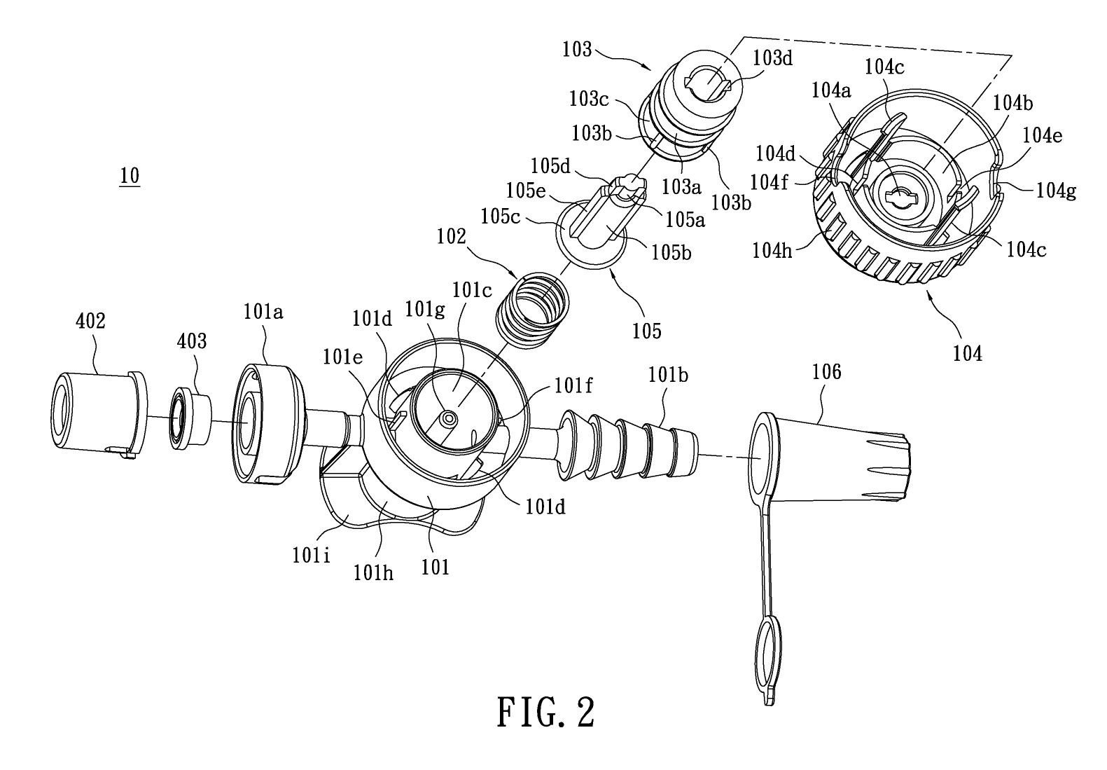

With reference to FIGS. 2˜5, a controller 10 for the suction catheter comprises a housing 101, an elastic member 102, a hollow valve body 103, which is referred to as the hollow valve body hereinafter, a actuating piston 105, and a cap 104. The elastic member 102, the actuating piston 105, and the hollow valve body 103 can be assembled in sequence inside the housing 101. Then, the cap 104 is disposed onto the housing 101 to complete the assembly. The assembling process can be easily accomplished to save manufacturing time.

A detailed description is given herein for the instant embodiment. Please refer to FIG. 2, which shows an exploded view of the controller 10. The housing 101 includes an inlet portion 101a and an axially extending tapered outlet portion 101b adapted for connection to a vacuum conduit (not shown). The housing 101 further includes a substantially coaxial tubular inner housing portion 101c, which is referred to as the inner housing portion hereinafter, is passingly bridging the inlet and outlet portions 101a and 101b. The outlet portion 101b can be connected to a vacuum generator (not shown) downstream, to provide the suction power. A dust cover 106 can be further disposed over the outlet portion 101b. A guiding post 101g projects from the housing 101 for inserting through the actuating piston 105.

The elastic member 102 can be tapered or shaped cylindrically for disposing inside the inner housing portion 101c. One end of the elastic member 102 abuts to the housing 101. The elastic member 102 can be a metallic or rubber coil spring. However, the shape and composition of the elastic member 102 is not restricted. For the instant embodiment, the elastic member 102 is a cylindrical metal spring. When the hollow valve body 103 is pressed downward, the elastic member 102, which is disposed in between the housing 101 and the cap 104, is actuated. In other words, the elastic member 102 provides the restoring energy for the hollow valve body 103.

Referring further to FIG. 2, a sealed end 101h is formed on the housing 101 away from the cap 104. Therefore, accidental contact of contaminated fluids can be prevented to reduce the risk of cross-contamination. The flanged surfaces (not labeled) reinforce the structural integrity of the sealed end 101h and saves manufacturing cost. The sealed end 101h has a base 101i with curved edges. Such design applies ergonomics practices for allowing the care giver to operate the controller 10 with less effort. For example, the care giver can hold the base 101i by the index finger and the ring finger, and put the thumb over the cap 104.

The hollow valve body 103 can be movably disposed inside the inner housing portion 101e. The actuating piston 105 projects through the hollow valve body 103, with one end of the actuating piston 105 abuts to the elastic member 102. The cap 104 is disposed on the housing 101. An opening 104a is formed on the cap 104 for engaging one end of the actuating piston 105 away from the elastic member 102. Structurally, the actuating piston 105 has a head portion 105a, a neck portion 105b, and a base portion 105c. At least one first ribbed portion 105d and at least one second ribbed portion 105e are disposed on the head portion 105a and the neck portion 105b respectively. Corresponding to the second ribbed portion 105e, at least one groove 103d is slotted axially on the inner surface of the hollow valve body 103. Likewise, the opening 104a of the cap 104 is formed for engaging the first ribbed portion 105d. To assemble the controller 10, as depicted in FIG. 2, the elastic member 102 can be first disposed inside the inner housing portion 101c. Next, the actuating piston 105 is secured to the hollow valve body 103 by engaging the second ribbed portion 105e with the groove 103d, wherein the actuating piston 105 engaged by the hollow valve body 103 abuts to the elastic member 102. The cap 104 is then fitted onto the housing 101, wherein the first ribbed portion 105d of the actuating piston 105 is engaged by the opening 104a. Finally, silicon oil can be added to the engaging location for lubrication and keeping air from entering the controller 10. For sealing, a sealing compound (not shown), such as hydrotreated heavy naphthenic, hydrocarbon, paraffin, or a mixture of at least two preceding compounds is applied over the engaging location to complete the assembling process. However, the choice of the sealing compound is not restricted.

As described in above, the first ribbed portion 105d and the second ribbed portion 105e of the actuating piston 105 are engaged to the opening 104a of the cap 104 and the groove 103d of the hollow valve body 103 respectively. Therefore, when the cap 104 is pressed and turned, the actuating piston 105 allows the hollow valve body 103 to move with the cap 104 in synchrony. The synchronized movement offsets potential slippage due to the silicon oil. Also, as already described earlier, one end of the actuating piston 105 is abutted to the elastic member 102. For the instant embodiment, the base portion 105c of the actuating piston 105 is disc-shaped. The elastic member 102 is a cylindrical coil spring. Thereby, the contact area between the base portion 105c and the elastic member 102 is greater. Versus conventional tapered spring, the controller 10 of the instant disclosure can better control the operation of the elastic member 102, and less effort is needed to actuate the elastic member 102. Furthermore, the cap 104 is not fixed to the housing 101. Thus, when the controller 10 is obsolete, only the sealing compound needs to be removed to disassemble the controller 10 for specialized medical waste disposal. Thereby, the potential risk of spreading the infection due to improper disassembling of the controller 10 can be greatly reduced.

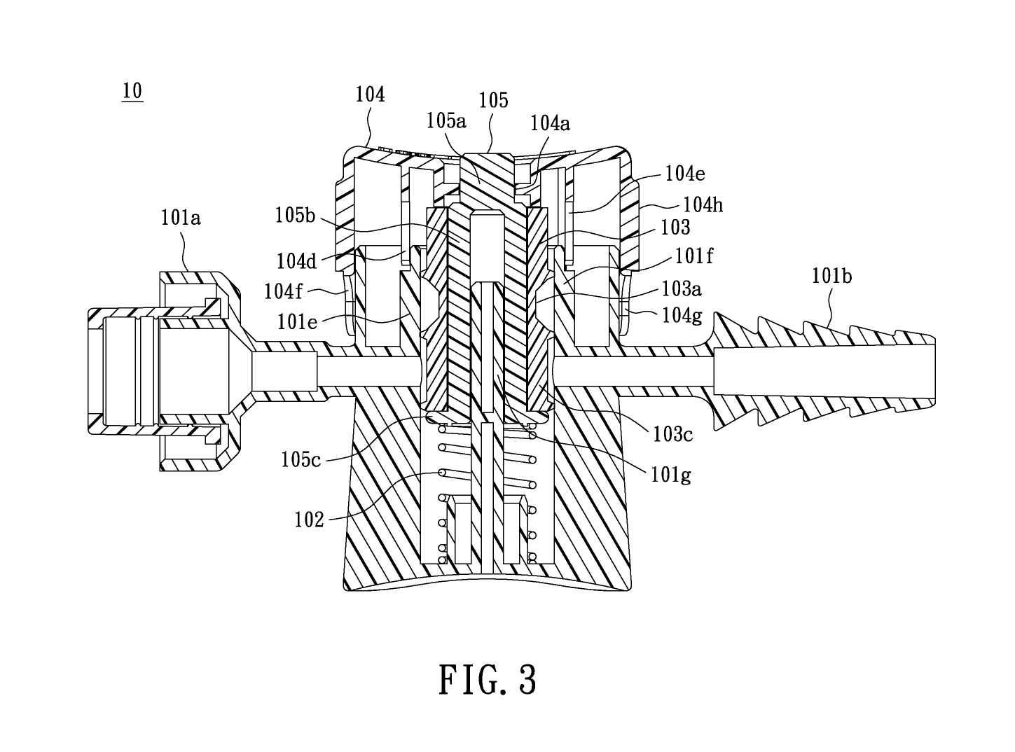

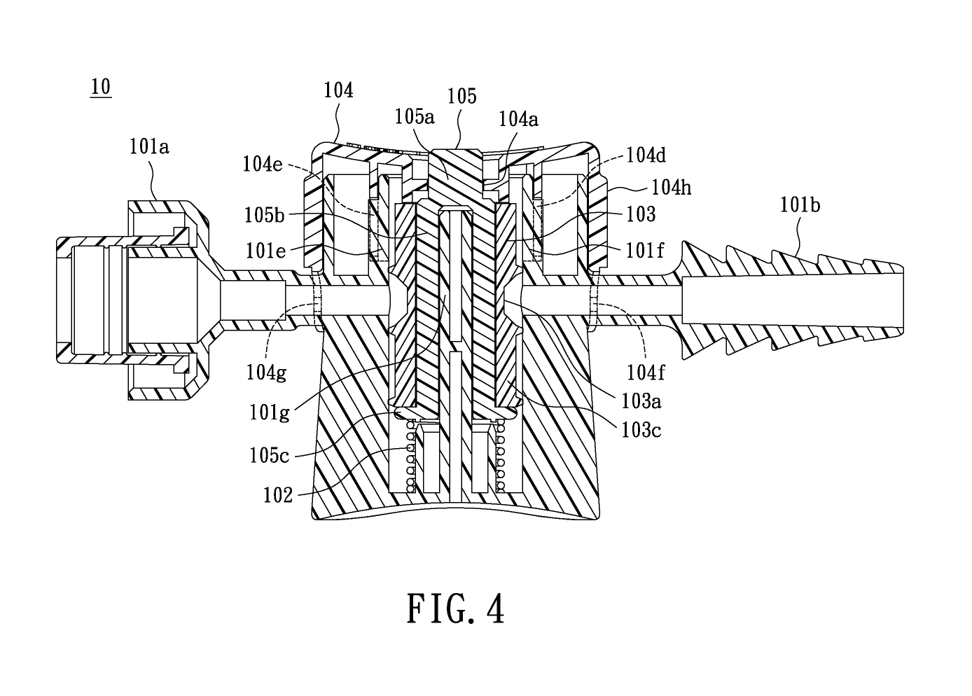

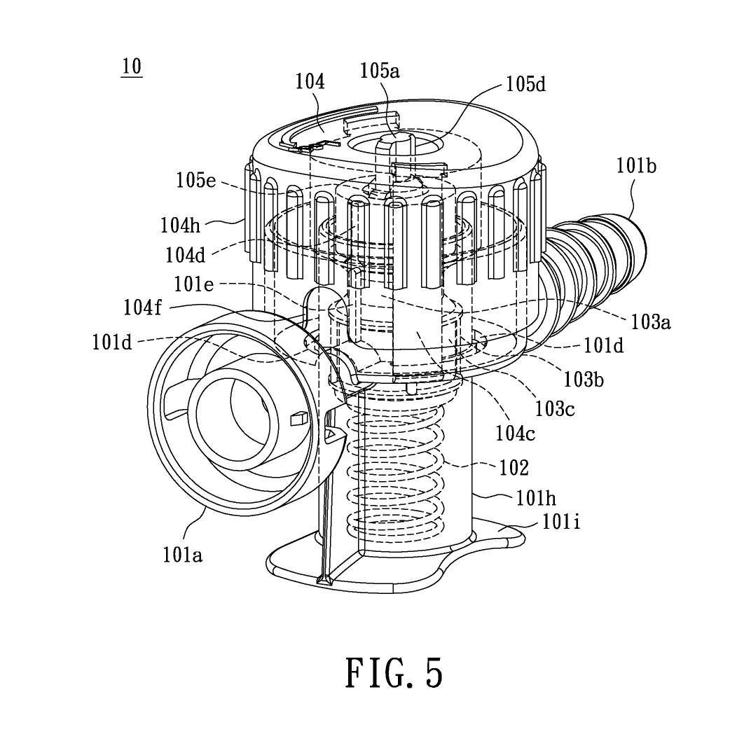

Regarding the operation of the controller 10, a detailed explanation is given herein. Please refer to FIGS. 3˜5. FIG. 3 shows a sectional view of the controller 10, wherein the hollow valve body 103 is biased to the closed position. FIG. 4 is for the controller 10 at the open position. FIG. 5 is a transparent view of the controller 10 showing the hollow valve body 103 at the closed position. As shown in FIGS. 3 and 4, the hollow valve body 103 can be movably disposed at the closed or open position. Referring back to FIG. 2, an annular groove 103a is formed on one end of the hollow valve body 103. On the opposite end, a plurality of vertical ribbed structures 103b are formed thereon and separated by a stop plate 103c in between each ribbed structure 103b. In other words, the ribbed structure 103b and the stop plates 103c are arranged together in intervals. Functionally, the ribbed structures 103b serve to strengthen the structural integrity of the hollow valve body 103 and its air tightness. As for the housing 101, at least one segmented guide slot 101d is formed circumferentially on the inner end surface thereof. Plus, at least one set block, such as a first set block 101e and a second set block 101f, are disposed on the outer surface of the inner housing portion 101c. For the cap 104 itself, an engaging portion 104b is disposed thereon. The engaging portion 104b has at least one extension 104c for engaging the guide slot 101d. At least one set groove, such as a first set groove 104d and a second set groove 104e, are formed on the engaging portion 104b to fit the first and second set blocks 101e and 101f respectively. For the outer surface of the cap 104, a first groove 104f and a second groove 104g are formed thereon for partially sleeving the inlet portion 101a and the outlet portion 101b respectively. In particular, the cap 104 can be turned clockwise or counter-clockwise. Also, the number of the aforementioned set blocks and the set grooves are not restricted. For the instant embodiment, two set blocks, namely the first and second set blocks 101e and 101f, and two set grooves, namely the first and second set grooves 104d and 104e, are illustrated for exemplary purpose.

Referring back to FIG. 3, which shows the hollow valve body 103 at closed position, when the cap 104 is not being pressed. Under such condition, the stop plates 103c block the flow from the inlet portion 101a to the outlet portion 101b.

FIG. 4 shows the hollow valve body 103 at the open position, wherein the cap 104 is pressed downward to actuate the hollow valve body 103. The extension 104c can be inserted thru the guide slot 101d (as shown in FIG. 5), and the cap 104 can be turned in the clockwise direction. When the extension 104c has traversed circumferentially for a predetermined distance along the guide slot 101d (as shown in FIG. 5), the first set block 101e and the second set block 101f of the housing 101 would engage to the second set groove 104e and the first set groove 104d respectively. Meanwhile, the first and second grooves 104f and 104g can be set over the outlet portion 101b and the inlet portion 101a respectively. Based on the above, the hollow valve body 103 can stay at the open position securely, wherein the annular groove 103a allows the flow between the inlet portion 101a and the outlet portion 101b for suctioning secretions. The care giver does not need to press and hold down the cap 104 continuously to perform the suctioning procedure. Thus, the care giver can save more effort and reduce the chance of muscle fatigue. Also, the care giver does not need to worry about the cap 104 being reversed inadvertently in causing stoppage during the suctioning and discomfort to the patient. Furthermore, a plurality of gripping members 104h can be disposed on the outer side surface of the cap 104. The gripping members 104h allow the care giver to have necessary friction in turning the cap 104 more easily, and making the use of the controller 10 more convenient.

When suctioning has been completed, the care giver can pull and turn the cap 104 in reverse (counter-clockwise direction), wherein the extension 104c would traverse circumferentially along the guide slot 101d, thereby returning the cap 104 to the original position. Accordingly, the hollow valve body 103 is returned to the closed position, wherein the stop plate 103c stops the flow from the inlet portion 101a to the outlet portion 101b for stopping the suction.

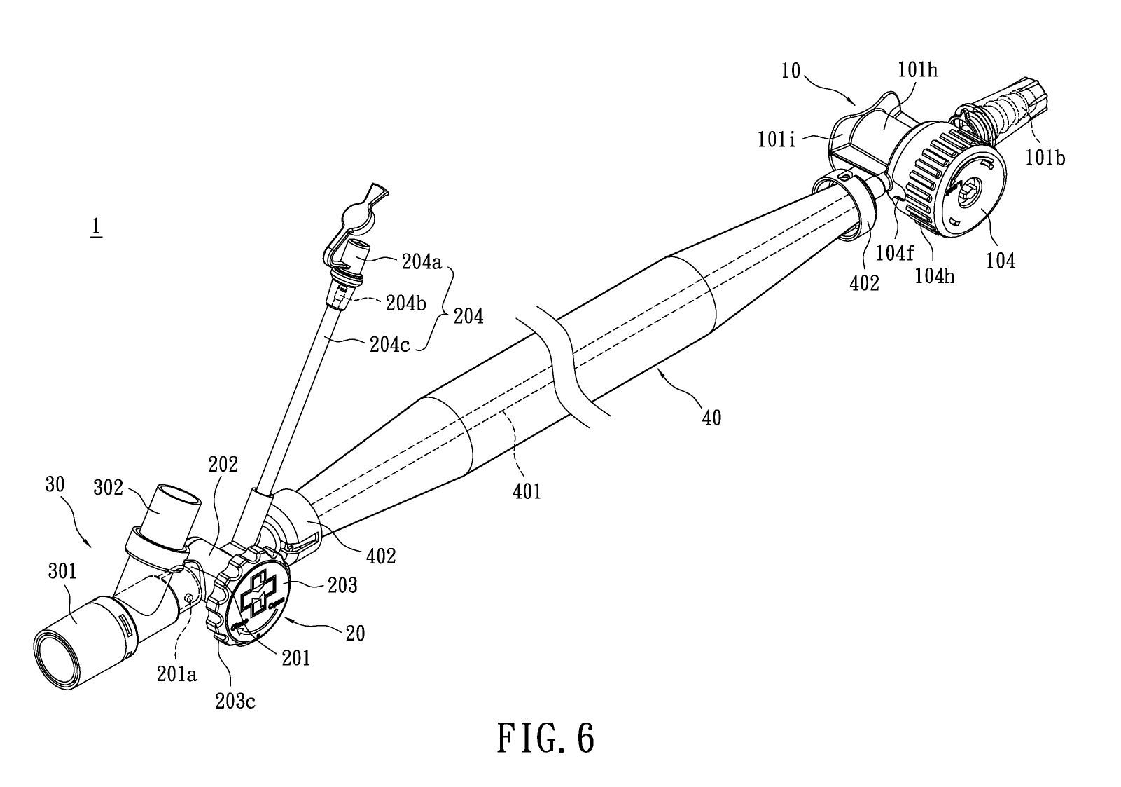

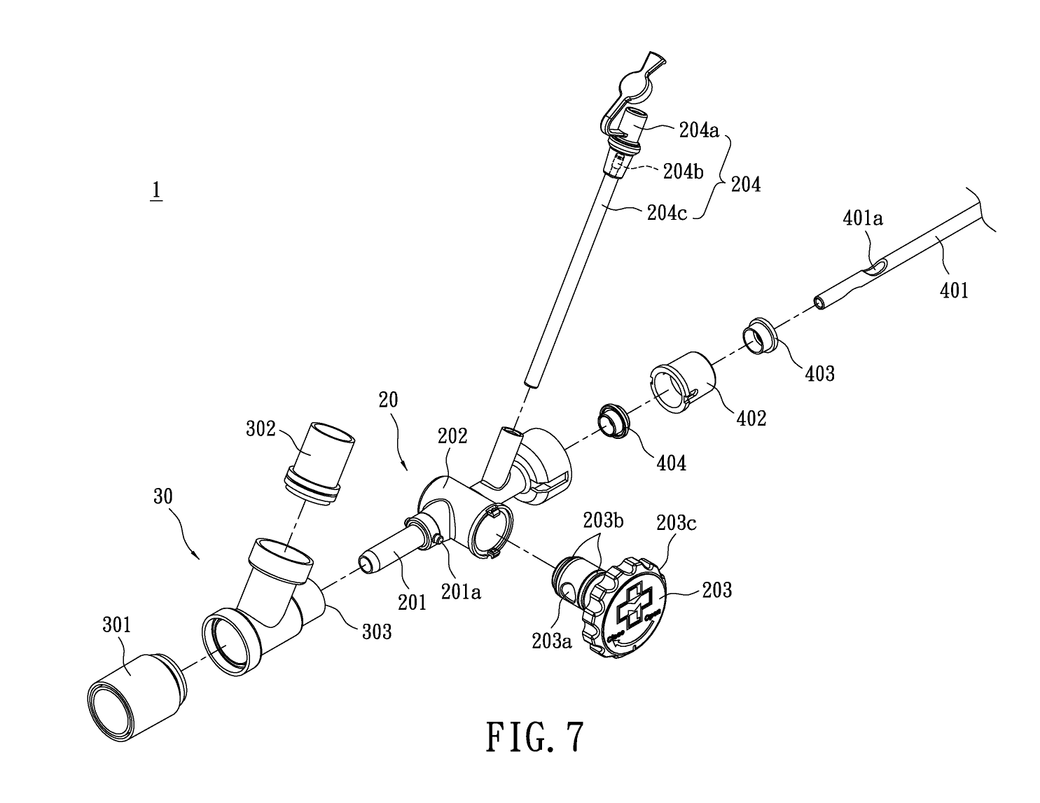

The instant disclosure also provides a suction catheter assembly 1, which is illustrated in FIGS. 6 and 7. FIG. 6 is an assembled view of the suction catheter assembly 1, and FIG. 7 is an exploded view for part thereof. As shown in FIG. 6, the suction catheter assembly 1 comprises the controller 10, a manifold member 20, an engaging structure 30, and a catheter 40. The controller 10 has already been described previously, therefore is not repeated herein. The manifold member 20 has a tubular arm 201 and a sleeve 202. The tubular arm 201 is passaged to the sleeve 202 perpendicularly. The engaging structure 30 is disposed at one end of the tubular arm 201. The catheter 40 is disposed at the opposite end of the tubular arm 201. A switch valve 203 is received by the sleeve 202, as shown in FIG. 7. The switch valve 203 has a thru hole 203a formed thereon and includes at least one o-ring 203b for sealing. The switch valve 203 is tapered for fitting to the sleeve 202 to enhance air tightness. The switch valve 203 can be turned to the open or closed position. At the open position, the thru hole 203a is in line with the tubular arm 201. At the closed position, the switch valve 203 blocks the fluid flow through the tubular arm 201. After the suction procedure has been completed, the switch valve 203 is normally turned to the closed position, thereby preventing the drained fluids from backflowing. Structurally, the switch valve 203 has a round valve handle (not labeled) for turning. A plurality of splined portions 203c is formed on the handle to prevent slipperiness. Thereby, the care giver can operate the switch valve 203 with one hand efficiently.

The engaging structure 30 is disposed at one end of the tubular arm 201. The engaging structure 30 comprises at least one connector 301 and at least one injection port 302. The connector 301 is for connecting an endotracheal tube (not shown), which can be inserted into the patient's airway. At least one engaging groove 303 is formed on the inner surface at one end of the engaging structure 30. In corresponding to the engaging groove 303, at least one tubular boss 201a is formed on the manifold member 20 and normal to the tubular arm 201. The tubular boss 201a can engage to the engaging groove 303 for connecting the engaging structure 30 at one end of the tubular arm 201. Furthermore, the connector 301 can be connected to a pulse oximeter (not shown) for monitoring a patient's oxygenation. A secretion container or remover (not shown) can also be disposed at the injecting port 302.

Notably, as shown in FIGS. 6 and 7, the aforementioned manifold member 20 has a hollow body. The engaging structure 30 is a three-way tubing, similar to a tee of the pipe fitting. Furthermore, an injection structure 204 can be disposed onto the manifold member 20. The injection structure 204 is for injecting secretion removal agent to clean the tubing. Structurally, the injection structure 204 has a tip 204a, a check valve 204b, and an injection tube 204c. The check valve 204b is disposed inside the tip 204a for preventing backflowing. The tip 204a is fitted onto a distal end of the injection tube 204c.

The catheter 40 comprises an elongated flexible catheter tube 401, two flanged bushings 402, two circular seal members 403, and a scrub ring 404. The flanged bushings 402 slide over the discharge end of the tubular arm 201 and the feeding end of the controller 10 respectively. Thus, the catheter 40 can be connected in between the tubular arm 201 and the controller 10. The catheter tube 401 is a long and narrow tube, which extends longitudinally through the flanged bushings 402, the engaging structure 30, the manifold member 20, and the inlet portion 101a. For the catheter tube 401, at least one lateral port 401a is formed therein and spaced a short distance from a distal end thereof. The catheter tube 401 can extend out of the breathing tube (not shown) into the patient's airway to drain the secretions. For the instant embodiment, the seal members 403 are made of rubber material, which covers the catheter tube partially to protect the care giver from contacting the secretions. As illustrated in FIG. 7, the scrub ring 404 is disposed in between the flanged bushing 402 and the tubular arm 201 for secretion removal. The scrub ring 404 is made of rubber grade material, but is not restricted thereto. Also, the catheter tube 401 can pass through the scrub ring 404.

Based on the above description, the advantages for the controller of the suction catheter and suction catheter assembly having the same of the instant disclosure are summarized below. First, when assembling the controller, only the elastic member, the actuating piston, and the hollow valve body need to be installed into the housing before disposing the cap. The assembling process is easy, which saves manufacturing time. Secondly, one end of the housing of the controller shrinks in forming a sealed end. The design is more cost effective and gives better air tightness. Thirdly, the controller, the actuating piston, the hollow valve body, and the cap give directional control in operating the controller. Plus, the actuating piston and the elastic member have greater contact area with each other versus conventional design. Thus, the elastic member can be controlled more effectively, with less effort required for pressing the controller. The cap and the housing of the controller are configured such that continuous suctioning is achieved, wherein the care giver does not need to hold down the controller continuously to perform the suctioning procedure. Thus, physical laboring and muscle fatigue can be reduced in operating the controller. The concern over accidental mishandling of the controller in causing drainage stoppage or patient discomfort is also eliminated. Furthermore, for the suction catheter assembly, the switch valve can be operated with one hand to facilitate the handling thereof.

The descriptions illustrated supra set forth simply the preferred embodiments of the instant disclosure; however, the characteristics of the instant disclosure are by no means restricted thereto. All changes, alternations, or modifications conveniently considered by those skilled in the art are deemed to be encompassed within the scope of the instant disclosure delineated by the following claims.

What is claimed is:

1. A suction catheter controller, comprising:
a housing having an inlet portion, an outlet portion, and an inner housing portion disposed in the housing communicatively bridging the inlet portion and the outlet portion;
an elastic member disposed in the inner housing portion abutting the inner surface of the housing on one end;
a hollow valve body disposed in the inner housing portion selectively movable between a closed position and an open position;
a actuating piston projected through the hollow valve body abutting the other end of the elastic member; and
a cap disposed on the housing having an opening formed on the top surface thereof for receiving one end of the actuating piston,
wherein the inlet portion is disabled from communicating with the outlet portion when the hollow valve body is at the closed position,
wherein the inlet portion is enabled to communicate with the outlet portion when the hollow valve body is at the open position.

2. The suction catheter controller of claim 1, wherein at least one segmented guide slot is formed circumferentially on the inner end surface of the housing, wherein at least one set block is disposed on the outer surface of the coaxial tubular inner housing portion, wherein the cap has an engaging portion having at least one extension for corresponding to the respective guide slot and at least one set groove for fitting the set block, and wherein a first groove and a second groove are formed on the outer side surface of the cap.

3. The suction catheter controller of claim 1, wherein the hollow valve body has an annular groove formed on one end thereof for communicating the inlet portion with the outlet portion at open position, and wherein the hollow valve body has a plurality of ribbed structures and stop plates formed on the opposite end thereof, the ribbed structures and stop plates are arranged in intervals, and wherein the inlet portion is blocked by the stop plates from communicating with the outlet portion when the hollow valve body is at closed position.

4. The suction catheter controller of claim 1, wherein the actuating piston has a head portion, a neck portion, and a base portion, wherein a first ribbed portion and a second ribbed portion are formed on the head portion and the neck portion respectively, wherein at least one groove is formed axially on the inner surface of the hollow valve body in corresponding to the second ribbed portion, and wherein the first ribbed portion engages matching to the opening of the cap.

5. The suction catheter controller of claim 1, wherein a guiding post is disposed on the inner end surface of the housing and inside the coaxial tubular inner housing portion for projecting through the actuating piston.

6. The suction catheter controller of claim 1, wherein a sealed end is formed on the housing opposite of the cap.

7. A suction catheter assembly, comprising:
a manifold having a tubular arm and a sleeve member communicatively joining thereto, the sleeve member including a switch valve having a thru hole formed thereon,
wherein the switch valve selectively operable to reach an open or a closed position,
wherein the thru hole is in line with the tubular arm at open position and blocks the tubular arm at closed position;
an engaging structure disposed at one end of the tubular arm, the engaging structure including at least one connector for connecting the endotracheal tube and at least one injection port passingly joining the connector;
a suction catheter disposed at the opposite end of the tubular arm away from the engaging structure; and
a suction catheter controller disposed at opposite end of the suction catheter away from the tubular arm, the controller of suction catheter including
a housing having an inlet portion, an outlet portion, and an inner housing portion disposed in the housing communicatively bridging the inlet portion and the outlet portion;
an elastic member disposed in the inner housing portion abutting the inner surface of the housing on one end;
a hollow valve body disposed in the inner housing portion selectively movable between a closed position and an open position;
a actuating piston projected through the hollow valve body abutting the other end of the elastic member; and
a cap disposed on the housing having an opening formed on the top surface thereof for receiving one end of the actuating piston,
wherein the inlet portion is disabled from communicating with the outlet portion when the hollow valve body is at the closed position,
wherein the inlet portion is enabled to communicate with the outlet portion when the hollow valve body is at the open position.

8. The suction catheter assembly of claim 7, wherein at least one segmented guide slot is formed circumferentially on the inner end surface of the housing, wherein at least one set block is disposed on the outer surface of the coaxial tubular inner housing portion, wherein the cap has an engaging portion having at least one extension for corresponding to the respective guide slot and at least one set groove for fitting the set block, and wherein a first groove and a second groove are formed on the outer side surface of the cap.

9. The suction catheter assembly of claim 7, wherein the hollow valve body has an annular groove formed on one end thereof for communicating the inlet portion with the outlet portion at open position, and wherein the hollow valve body has a plurality of ribbed structures and stop plates formed on the opposite end thereof, the ribbed structures and stop plates are arranged in intervals, and wherein the inlet portion is blocked by the stop plates from communicating with the outlet portion when the hollow valve body is at closed position.

10. The suction catheter assembly of claim 7, wherein the actuating piston has a head portion, a neck portion, and a base portion, wherein a first ribbed portion and a second ribbed portion are formed on the head portion and the neck portion respectively, wherein at least one groove is formed axially on the inner surface of the hollow valve body in corresponding to the second ribbed portion, and wherein the first ribbed portion engages matching to the opening of the cap.

11. The suction catheter assembly of claim 7, wherein a sealed end is formed on the housing opposite of the cap.

12. The suction catheter assembly of claim 7, wherein the elastic member is shaped cylindrically.

13. The suction catheter assembly of claim 7, wherein at least one injection structure is disposed on the tubular arm, and wherein the injection structure comprises an injection tube, a check valve, and a tip, the check valve being disposed inside the tip, the tip being sleeved onto the injection tube.

14. The suction catheter assembly of claim 7, wherein at least one engaging groove is formed circumferentially on one end of the engaging structure, wherein at least one tubular boss is disposed on the tubular arm for disposing inside the corresponding engaging groove, and wherein an endotracheal tube can be attached to the connector.

15. The suction catheter assembly of claim 7, wherein a handle of the switch valve is disc-shaped having a plurality of splined portions for reducing slipperiness.

16. The suction catheter assembly of claim 7, wherein the suction catheter comprises a catheter tube, two flanged bushings, and two circular seal members, wherein the flanged bushings sleeve over one end of the tubular arm and the front end of the inlet portion, the catheter tube being projected through the flanged bushings, wherein the circular seal members cover the catheter tube partially.

17. The suction catheter assembly of claim 16, wherein a scrub ring is disposed in between the tubular arm and the flanged bushing of the catheter tube for removing the secretions, and wherein the catheter tube projects through the scrub ring.

18. The suction catheter assembly of claim 16, wherein at least one lateral port is formed on the catheter tube and near one end thereof.
